(19) United States
(12) Patent Application Publication
(10) Pub. No.: US 2024/0350100 A1
(43) Pub. Date: Oct. 24, 2024

(54) METHOD AND SYSTEM FOR GENERATING RESPIRATION SIGNALS FOR USE IN ELECTROPHYSIOLOGY PROCEDURES

(71) Applicant: St. Jude Medical, Cardiology Division, Inc., St. Paul, MN (US)

(72) Inventors: Hai Huang, Woodbury, MN (US); Orit Brimer Limor, Haifa (IS); Eric Olson, Maplewood, MN (US)

(21) Appl. No.: 18/682,349

(22) PCT Filed: Aug. 24, 2022
(86) PCT No.: PCT/US22/41362

§ 371 Date: Feb. 8, 2024

Related U.S. Application Data
(60) Provisional application No. 63/237,269, filed on Aug. 26, 2021.

(51) Int. Cl. A61B 5/00; A61B 5/367

(52) U.S. Cl. CPC A61B 5/7289; A61B 5/367

(57) ABSTRACT

A respiration signal can be generated within electroanatomical mapping system from the non-driven impedance signals received from a plurality of patch electrodes. The non-driven impedance signals are used to define a reference respiration signal. Each of a subset of the non-driven impedance signals can then be compared to the reference respiration signal to determine a polarity value; a scaling factor can also be computed that normalizes the non-driven impedance signals. The polarity values and scaling factors are applied to the non-driven impedance signals to generate weighted non-driven impedance signals, which can then be summed into a composite respiration signal. The composite respiration signal can, in turn, be subject to its own polarity value and scaling factor for use in real time (e.g., for gating data collection, respiration compensation, detection of irregular respiration, and the like).

### CROSS-REFERENCE TO RELATED APPLICATIONS

This application claims the benefit of U.S. provisional application No. 63/237,269, filed 26 Aug. 2021, which is hereby incorporated by reference as though fully set forth herein.

### BACKGROUND

The present disclosure relates generally to electrophysiology procedures, such as cardiac diagnostic and therapeutic procedures, including electrophysiological mapping and cardiac ablation. In particular, the present disclosure relates to the generation of composite respiration signals for use during such electrophysiology procedures.

It is known to use respiration signals in connection with various electrophysiology procedures. For example, respiration signals can be used for gating, to detect irregular respiration, and to compensate for certain motion during electrophysiological mapping (such as described in U.S. Pat. No. 7,263,397, which is hereby incorporated by reference as though fully set forth herein).

In many extant electroanatomical mapping systems, respiration signals are derived from impedance measurements at one or more body surface (e.g., patch) electrodes. Due to variations in the placement of such electrodes, however, as well as variations in environmental and physiological conditions, these impedance signals can exhibit certain shortcomings, including baseline wander, low amplitude, or inverted polarity. These shortcomings can lead to incorrect gating, false positive or false negative detection of irregular respiration, and errors in respiration motion compensation algorithms. This, in turn, can lead to electrophysiology catheter motion artifacts.

### BRIEF SUMMARY

The instant disclosure provides a method of generating a respiration signal within an electroanatomical mapping system. According to aspects of the disclosure, the method includes the electroanatomical mapping system: receiving a plurality of non-driven impedance signals from a plurality of patch electrodes; defining a reference respiration signal using the plurality of non-driven impedance signals; for each non-driven impedance signal within a subset of the plurality of non-driven impedance signals: computing a polarity value for the non-driven impedance signal; and computing a scaling factor for the non-driven impedance signal; and computing a composite respiration signal from the subset of the plurality of non-driven impedance signals.

In embodiments of the disclosure, computing the polarity value for the non-driven impedance signal includes: computing a correlation coefficient between the non-driven impedance signal and the reference respiration signal; and computing the polarity value based on a sign of the correlation coefficient.

In other embodiments of the disclosure, computing the scaling factor for the non-driven impedance signal includes normalizing the non-driven impedance signal, such as by dividing the non-driven impedance signal by its signal range.

The composite respiration signal can be computed from the subset of the plurality of non-driven impedance signals by: multiplying each non-driven impedance signal within the subset of the plurality of non-driven impedance signals by its corresponding polarity value and scaling factor, thereby computing a plurality of weighted non-driven impedance signals; and computing the composite respiration signal by summing the plurality of weighted non-driven impedance signals.

It is also contemplated to define a polarity value of the composite respiration signal. For example, the polarity value of the composite respiration signal can be defined such that a polarity of the composite respiration signal corresponds to a polarity of a PRS-A signal. Alternatively, the polarity value of the composite respiration signal is defined to correspond to the polarity of the PRS-A signal only when a correlation coefficient between the composite respiration signal and the PRS-A signal exceeds a preset threshold, such as about 75%.

In still other embodiments of the disclosure, the polarity value of the composite respiration signal can be defined according to an assumption that a duration of expiration in the composite respiration signal exceeds a duration of inspiration in the composite respiration signal. For instance, the polarity value of the composite respiration signal can be defined such that a minimum of the composite respiration signal is closer to a mean of the composite respiration signal than a maximum of the composite respiration signal is to the mean of the composite respiration signal.

As another example, the polarity value of the composite respiration signal can be defined such that a mean of troughs in the composite respiration signal is closer to a mean of the composite respiration signal than a mean of peaks in the composite respiration signal is to the mean of the composite respiration signal.

As yet another example, the polarity value of the composite respiration signal can be defined such that a time interval between an earlier downward zero crossing and a later upward zero crossing in the composite respiration signal is longer than a time interval between an earlier upward zero crossing and a later downward zero crossing in the composite respiration signal.

A composite scaling factor for the composite respiration signal that normalizes the composite respiration signal can also be computed.

The step of defining the reference respiration signal using the plurality of non-driven impedance signals can include defining either a strongest non-driven impedance signal of the plurality of impedance signals or a first principal component signal of the plurality of non-driven impedance signals as the reference respiration signal.

The strongest non-driven impedance signal of the plurality of non-driven impedance signals can be a largest standard deviation signal of the plurality of non-driven impedance signals, a highest amplitude signal of the plurality of non-driven impedance signals, or another suitable signal.

Similarly, the step of defining either the strongest non-driven impedance signal of the plurality of impedance signals or the first principal component signal of the plurality of non-driven impedance signals as the reference respiration signal can include defining either the strongest impedance signal of the plurality of non-driven impedance signals or the first principal component signal of the plurality of non-driven impedance signals as the reference respiration signal according to correlation coefficients between the strongest impedance signal and the subset of the plurality of the non-driven impedance signals, on one hand, and between the first principal component signal and the subset of the plurality of the non-driven impedance signals, on the other hand.

The instant disclosure also provides an electroanatomical mapping system, including a respiration compensation module that is configured to: receive a plurality of non-driven impedance signals from a plurality of patch electrodes; define a reference respiration signal using the plurality of non-driven impedance signals; for each non-driven impedance signal within a subset of the plurality of non-driven impedance signals: compute a polarity value for the non-driven impedance signal; and compute a scaling factor for the non-driven impedance signal; and compute a composite respiration signal from the subset of the plurality of non-driven impedance signals by multiplying each non-driven impedance signal within the subset of the plurality of non-driven impedance signals by its corresponding polarity value and scaling factor, thereby computing a plurality of weighted non-driven impedance signals, and summing the plurality of weighted non-driven impedance signals.

The respiration compensation module can define the reference respiration signal as either a strongest non-driven impedance signal of the plurality of impedance signals or a first principal component signal of the plurality of non-driven impedance signals as the reference respiration signal.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 depicts an exemplary catheter that can be used in connection with aspects of the instant disclosure.

FIGS. 3A and 3B provide alphanumeric labeling conventions for electrodes carried by a multi-electrode catheter and the bipoles associated therewith.

FIG. 4 is a flowchart of representative steps that can be carried out according to aspects of the instant disclosure.

FIG. 5 is a representative composite respiration signal according to aspects disclosed herein.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for generating respiration signals for use in an electrophysiology procedure. For purposes of illustration, aspects of the disclosure will be described with reference to an electrophysiology study carried out using a high density (HD) grid catheter, such as the Advisor™ HD Grid Mapping Catheter, Sensor Enabled™, from Abbott Laboratories (Abbott Park, Illinois), in conjunction with an electroanatomical mapping system, such as the EnSite Precision™ cardiac mapping system or the Ensite™ X EP System, both also from Abbott Laboratories. Those of ordinary skill in the art will understand, however, how to apply the teachings herein to good advantage in other contexts and/or with respect to other devices.

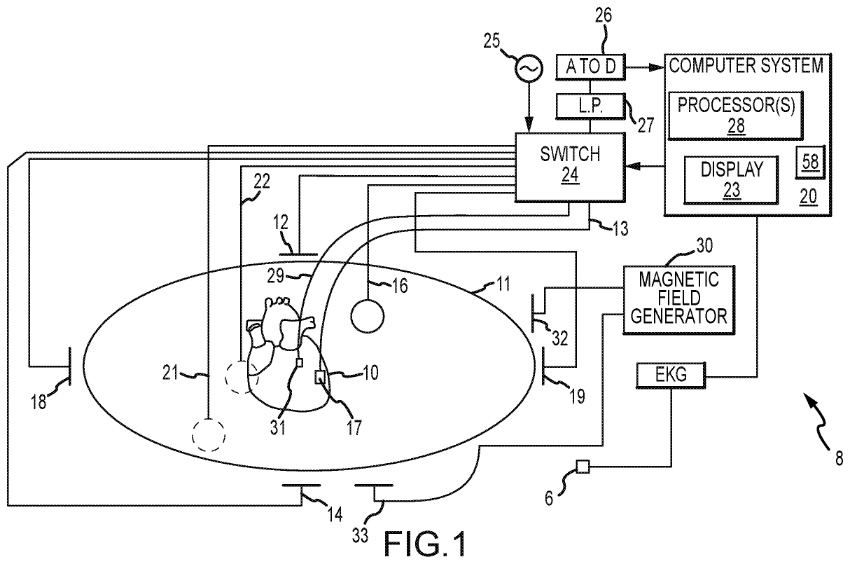

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference. This is referred to herein as “localization.”

For simplicity of illustration, the patient 11 is depicted schematically as an oval. In the embodiment shown in FIG. 1, three sets of surface electrodes (e.g., patch electrodes) 12, 14, 16, 18, 19, and 22 are shown applied to a surface of the patient 11, pairwise defining three generally orthogonal axes, referred to herein as an x-axis (12, 14), a y-axis (18, 19), and a z-axis (16, 22). In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

According to embodiments of the instant disclosure, each surface electrode measures six signals-three resistance (impedance) signals and three reactance signals. These signals can, in turn, be grouped into three resistance/reactance signal pairs. One resistance/reactance signal pair reflects driven values, as described below, while the other two resistance/reactance signal pairs reflect non-driven values (e.g., measurements of the electric field generated by other driven pairs in a manner similar to that described below for electrodes 17).

An additional surface reference electrode (e.g., a “belly patch”) 21 provides a reference and/or ground electrode for the system 8. The belly patch electrode 21 may be an alternative to a fixed intra-cardiac electrode 31, described in further detail below. In alternative embodiments, the surface reference electrode 21 can alternatively or additionally include a magnetic patient reference sensor-anterior (“PRS-A”) positioned on the patient's chest.

It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes.

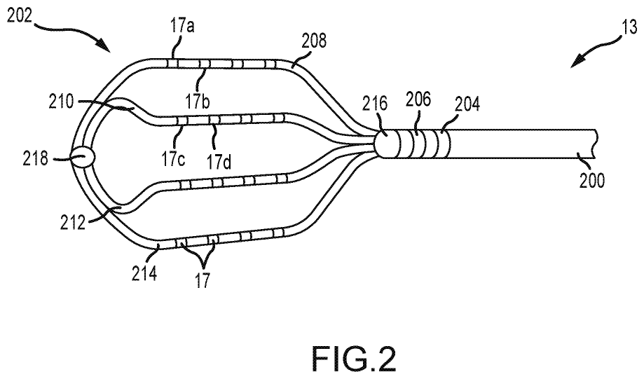

The foregoing embodiments are merely exemplary, however, and any number of electrodes and/or catheters may be used. For example, for purposes of this disclosure, a segment of an exemplary multi-electrode catheter, and in particular an HD grid catheter 13 such as the Advisor™ HD Grid Mapping Catheter, Sensor Enabled™, is shown in FIG. 2. HD grid catheter 13 includes a catheter body 200 coupled to a paddle 202. Catheter body 200 can further include first and second body electrodes 204, 206, respectively. Paddle 202 can include a first spline 208, a second spline 210, a third spline 212, and a fourth spline 214, which are coupled to catheter body 200 by a proximal coupler 216 and to each other by a distal coupler 218. In one embodiment, first spline 208 and fourth spline 214 can be one continuous segment and second spline 210 and third spline 212 can be another continuous segment. In other embodiments, the various splines 208, 210, 212, 214 can be separate segments coupled to each other (e.g., by proximal and distal couplers 216, 218, respectively). It should be understood that HD catheter 13 can include any number of splines; the four-spline arrangement shown in FIG. 2 is merely exemplary.

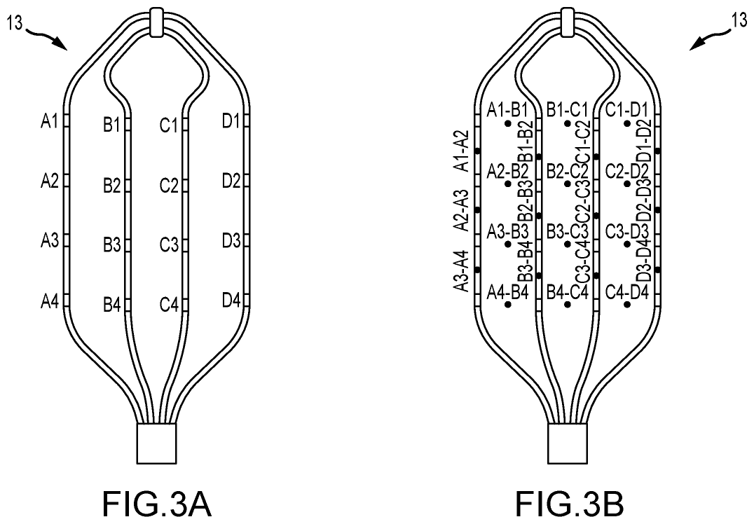

As described above, splines 208, 210, 212, 214 can include any number of electrodes 17; in FIG. 2, sixteen electrodes 17 are shown arranged in a four-by-four array. It should also be understood that electrodes 17 can be evenly and/or unevenly spaced, as measured both along and between splines 208, 210, 212, 214. For purposes of easy reference in this description, FIG. 3A provides alphanumeric labels for electrodes 17.

As those of ordinary skill in the art will recognize, any two neighboring electrodes 17 define a bipole. Thus, the 16 electrodes 17 on catheter 13 define a total of 42 bipoles—12 along splines (e.g., between electrodes 17a and 17b, or between electrodes 17c and 17d), 12 across splines (e.g., between electrodes 17a and 17c, or between electrodes 17b and 17d), and 18 diagonally between splines (e.g., between electrodes 17a and 17d, or between electrodes 17b and 17c).

For ease of reference in this description, FIG. 3B provides alphanumeric labels for the along- and across-spline bipoles. FIG. 3B omits alphanumeric labels for the diagonal bipoles, but this is only for the sake of clarity in the illustration. It is expressly contemplated that the teachings herein can also be applied with respect to the diagonal bipoles.

Any bipole can, in turn, be used to generate a bipolar electrogram according to techniques that will be familiar to those of ordinary skill in the art. Moreover, these bipolar electrograms can be combined (e.g., linearly combined) to generate electrograms, again including activation timing information, in any direction of the plane of catheter 13 by computing an E-field loop for a clique of electrodes. United States patent application publication no. 2018/0296111 (the '111 publication), which is hereby incorporated by reference as though fully set forth herein, discloses details of computing an E-field loop for a clique of electrodes on a HD grid catheter. These electrograms are referred to herein as “omnipolar electrograms,” and their corresponding directions are referred to herein as “omnipoles” or “virtual bipoles.”

In any event, catheter 13 can be used to simultaneously collect a plurality of electrophysiology data points for the various bipoles defined by electrodes 17 thereon, with each such electrophysiology data point including both localization information (e.g., position and orientation of a selected bipole) and an electrogram signal for the selected bipole. For purposes of illustration, methods according to the instant disclosure will be described with reference to individual electrophysiology data points collected by catheter 13. It should be understood, however, that the teachings herein can be applied, in serial and/or in parallel, to multiple electrophysiology data points collected by catheter 13.

Catheter 13 (or multiple such catheters) are typically introduced into the heart and/or vasculature of the patient via one or more introducers and using familiar procedures. Indeed, various approaches to introduce catheter 13 into a patient's heart, such as transseptal approaches, will be familiar to those of ordinary skill in the art, and therefore need not be further described herein.

Since each electrode 17 lies within the patient, location data may be collected simultaneously for each electrode 17 by system 8. Similarly, each electrode 17 can be used to gather electrophysiological data from the cardiac surface (e.g., endocardial electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate a graphical representation of a cardiac geometry and/or of cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., by driving pairs of patch electrodes 12/14, 18/19, and 16/22) in order to realize catheter navigation in a biological conductor. Alternatively, these orthogonal fields can be decomposed and any pairs of patch electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two patch electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are likewise exposed to the field from a current pulse and can likewise be measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 8 can be a hybrid system that incorporates both impedance-based (e.g., as described above) and magnetic-based localization capabilities. Thus, for example, system 8 can also include a magnetic source 30, which is coupled to one or more magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrode sets 12, 14, 16, 18, 19, and 22) can be used without departing from the scope of the present teachings. Likewise, those of ordinary skill in the art will appreciate that, for purposes of localizing catheter 13 within the magnetic fields so generated, catheter 13 can include one or more magnetic localization sensors (e.g., coils).

In some embodiments, system 8 is the EnSite™ Velocity™, EnSite Precision™, or EnSite™ X cardiac mapping and visualization system of Abbott Laboratories. Other localization systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stereotaxis, Inc.'s NIOBE® Magnetic Navigation System (St. Louis, Missouri), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to generating composite respiration signals for use by system 8 (e.g., for respiration gating, motion compensation, and/or detection of irregular respiration). System 8 can therefore include a respiration compensation module 58.

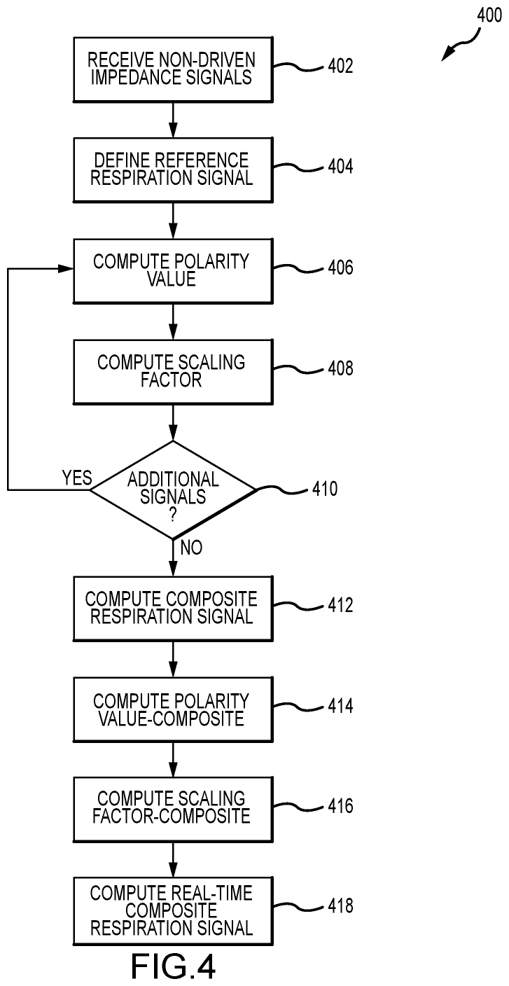

One exemplary method according to aspects of the instant disclosure will be explained with reference to the flowchart 400 of representative steps presented as FIG. 4. In some embodiments, for example, flowchart 400 may represent several exemplary steps that can be carried out by electroanatomical mapping system 8 of FIG. 1 (e.g., by processor 28 and/or respiration compensation module 58). It should be understood that the representative steps described below can be either hardware- or software-implemented. For the sake of explanation, the term “signal processor” is used herein to describe both hardware- and software-based implementations of the teachings herein.

In block 402, a plurality of non-driven impedance signals are received from patch electrodes 12, 14, 16, 18, 19, and 22. In particular, it is desirable to use the non-driven impedance signals from the chest, back, left side, right side, and neck electrodes; the non-driven signals from the left leg electrode typically are not used according to the instant teachings. Likewise, insofar as reactance signals are more sensitive to disturbance, the non-driven reactance signals will generally be ignored when applying the instant teachings.

A reference respiration signal (also called a base respiration signal) is defined in block 404, typically using the non-driven impedance signals measured by body surface electrodes 12, 14, 16, 19, 22 (e.g., excluding left leg electrode 18) received in block 402. Various approaches to defining the reference respiration signal are contemplated.

According to certain embodiments of the disclosure, the strongest non-driven impedance signal of the plurality of non-driven impedance signals received in block 402 is defined as the reference respiration signal. Those of ordinary skill in the art will appreciate that the term “strongest non-driven impedance signal” can be defined in various ways, including, by way of example only, the non-driven impedance signal with the largest standard deviation or the non-driven impedance signal with the highest amplitude.

In other embodiments of the disclosure, the first principal component signal of the plurality of non-driven impedance signals received in block 402, which may be computed using singular value decomposition, is defined as the reference respiration signal.

It is contemplated that the choice between using the strongest non-driven impedance signal and the first principal component signal as the reference respiration signal may be based on correlation coefficients. That is, once the strongest non-driven impedance signal has been identified, correlation coefficients can be computed between the strongest non-driven impedance signal and the remaining non-driven impedance signals received in block 402. The mean of the absolute value of these correlation coefficients can also be computed.

Likewise, once the first principal component signal has been computed, correlation coefficients can be computed between the first principal component signal and the non-driven impedance signals received in block 402. Once again, the mean of the absolute value of these correlation coefficients can also be computed. Whichever signal as between the strongest non-driven impedance signal and the first principal component approach yields the larger mean absolute value of correlation coefficients can be chosen as the reference respiration signal.

In block 406, a polarity value is computed for a selected one of the non-driven impedance signals received in block 402. The polarity value is used to align the selected non-driven impedance signal with the reference respiration signal, and will typically be +1 if the selected non-driven impedance signal has a positive correlation to the reference respiration signal and −1 if the selected non-driven impedance signal has a negative correlation to the reference respiration signal.

In block 408, a scaling factor is computed for the selected non-driven impedance signal. The scaling factor normalizes the selected non-driven impedance signal, for example by dividing the selected non-driven impedance signal by its signal range.

Block 410 initiates a loop back to blocks 406, 408 to compute polarity values and scaling factors for any additional non-driven impedance signals. It should be understood that not all non-driven impedance signals need be used, and that it is expressly contemplated that fewer than all non-driven impedance signals may be used. In particular, aspects of the disclosure utilize only the non-driven signals from the left side, right side, anterior, and posterior patch electrodes, excluding the neck and left leg patch electrodes.

If there are no additional non-driven impedance signals to process, then the “NO” exit from block 410 leads to block 412, where a composite respiration signal is computed. In particular, each non-driven impedance signal is multiplied by its polarity value and scaling factor, thus computing a corresponding weighted non-driven impedance signal. All weighted non-driven impedance signals are then summed to compute the composite respiration signal. Expressed mathematically, this is:

$$Composite ( t ) = {\sum }_{i} ( {Signal}_{i} ( t ) * {Polarity}_{i} * {Scaling}_{i} ) ,$$

where Signalᵢ(t) is the i^{th} non-driven impedance signal, Polarityᵢ is its corresponding polarity value, and Scaling, is its corresponding scaling factor.

In block 414, a polarity value is computed for the composite respiration signal (referred to herein as a “polarity value-composite”). Various approaches are contemplated.

For instance, in embodiments of the disclosure where a PRS-A signal is available, the polarity value-composite may be computed based on the correlation between the composite respiration signal and the PRS-A signal. More particularly, if the absolute correlation between the composite respiration signal and the PRS-A signal exceeds a preset threshold, such as about 75%, the polarity value-composite will be defined such that the polarity of the composite respiration signal is the same as the polarity of the PRS-A signal (e.g., +1 if the correlation is positive and −1 if the correlation is negative).

If a PRS-A signal is not available, or if the correlation between the composite respiration signal and the PRS-A signal does not exceed the preset threshold, then various heuristics may be employed to compute the polarity value-composite. In particular, the polarity value-composite may be defined according to the assumption that, within the composite respiration signal, the expiration phase is longer than the inspiration phase. Thus, the polarity value-composite may be defined (e.g., as either +1 or −1), such that one or more of the following conditions are true:
The minimum of the composite respiration signal is closer to the mean of the composite respiration signal than the maximum of the composite respiration signal;
The mean of troughs in the composite respiration signal is closer to a mean of the composite respiration signal than the mean of peaks in the composite respiration signal; or
A time interval between an earlier downward zero crossing and a later upward zero crossing in the composite respiration signal is longer than a time interval between an earlier upward zero crossing and a later downward zero crossing in the composite respiration signal.

In block 416, a scaling factor is computed for the composite respiration signal (i.e., “scaling factor-composite”). Similar to the scaling factors computed in block 408, the scaling factor-composite normalizes the composite respiration signal, such as by dividing the composite respiration signal by its signal range.

Once the polarity value-composite and scaling factor-composite have been computed, the composite respiration signal at any given time t (that is, the real-time composite respiration signal) can be computed (block 418) as the sum of all weighted non-driven impedance signals at time t, multiplied by polarity value-composite and scaling factor-composite. Expressed mathematically, this is:

$$Composite ( t ) = {Polarity}_{c o m p} * {Scaling}_{c o m p} * {\sum }_{i} ( {Signal}_{i} ( t ) * {Polarity}_{i} * {Scaling}_{i} ) ,$$

where Polarity_{comp} is the polarity value-composite and Scaling comp is the scaling factor-composite.

This real-time composite respiration signal can, in turn, be used for any desirable purpose (e.g., gating the collection of electrophysiological data, respiration compensation, detection of irregular respiration, and the like).

It is also contemplated that the real-time composite respiration signal may be high-pass filtered prior to use, e.g., using a filter with a cutoff frequency of about 0.02 Hz. One suitable high-pass filter cascades together two exponential moving average filters of the form:

$$y [ n ] = a * x [ n ] + ( 1 - a ) * y [ n - 1 ] ,$$

where a is about 0.002. A high-pass filter may, however, be disadvantageous if there are rapid changes in amplitude in the real-time composite respiration signal. Accordingly, in alternative embodiments of the disclosure, the real-time composite respiration signal is further normalized, and in particular shifted, to the end of its expiration phase. This can be achieved, for example, by subtracting the running minimum of the composite respiration signal from the composite respiration signal.

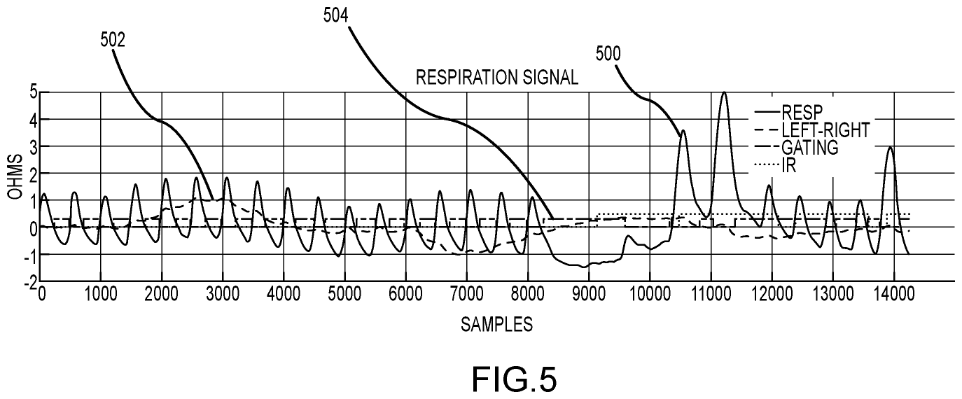

FIG. 5 depicts a composite respiration signal 500 according to the foregoing teachings. For the sake of comparison, FIG. 5 also illustrates a respiration signal derived from the driven impedance signals from the left side and right side patch electrodes according to extant respiration signal approaches. Finally, FIG. 5 depicts a gating signal 504 based on respiration signal 500.

Although several embodiments have been described above with a certain degree of particularity, those skilled in the art could make numerous alterations to the disclosed embodiments without departing from the spirit or scope of this invention.

For example, the teachings herein can be applied in real time (e.g., during an electrophysiology study) or during post-processing (e.g., to electrophysiology data points collected during an electrophysiology study performed at an earlier time).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of generating a respiration signal within an electroanatomical mapping system, comprising the electroanatomical mapping system:
receiving a plurality of non-driven impedance signals from a plurality of patch electrodes;
defining a reference respiration signal using the plurality of non-driven impedance signals;
for each non-driven impedance signal within a subset of the plurality of non-driven impedance signals:
computing a polarity value for the non-driven impedance signal; and
computing a scaling factor for the non-driven impedance signal; and
computing a composite respiration signal from the subset of the plurality of non-driven impedance signals.

2. The method according to claim 1, wherein computing the polarity value for the non-driven impedance signal comprises:
computing a correlation coefficient between the non-driven impedance signal and the reference respiration signal; and
computing the polarity value based on a sign of the correlation coefficient.

3. The method according to claim 1, wherein computing the scaling factor for the non-driven impedance signal comprises normalizing the non-driven impedance signal.

4. The method according to claim 3, wherein normalizing the non-driven impedance signal comprises dividing the non-driven impedance signal by its signal range.

5. The method according to claim 1, wherein computing the composite respiration signal from the subset of the plurality of non-driven impedance signals comprises:
multiplying each non-driven impedance signal within the subset of the plurality of non-driven impedance signals by its corresponding polarity value and scaling factor, thereby computing a plurality of weighted non-driven impedance signals; and
computing the composite respiration signal by summing the plurality of weighted non-driven impedance signals.

6. The method according to claim 1, further comprising defining a polarity value of the composite respiration signal.

7. The method according to claim 6, wherein the polarity value of the composite respiration signal is defined such that a polarity of the composite respiration signal corresponds to a polarity of a PRS-A signal.

8. The method according to claim 7, wherein the polarity value of the composite respiration signal is defined such that the polarity of the composite respiration signal corresponds to the polarity of the PRS-A signal only when a correlation coefficient between the composite respiration signal and the PRS-A signal exceeds a preset threshold.

9. The method according to claim 8, wherein the preset threshold is 75%.

10. The method according to claim 6, wherein the polarity value of the composite respiration signal is defined according to an assumption that a duration of expiration in the composite respiration signal exceeds a duration of inspiration in the composite respiration signal.

11. The method according to claim 10, wherein the polarity value of the composite respiration signal is defined such that a minimum of the composite respiration signal is closer to a mean of the composite respiration signal than a maximum of the composite respiration signal is to the mean of the composite respiration signal.

12. The method according to claim 10, wherein the polarity value of the composite respiration signal is defined such that a mean of troughs in the composite respiration signal is closer to a mean of the composite respiration signal than a mean of peaks in the composite respiration signal is to the mean of the composite respiration signal.

13. The method according to claim 10, wherein the polarity value of the composite respiration signal is defined such that a time interval between an earlier downward zero crossing and a later upward zero crossing in the composite respiration signal is longer than a time interval between an earlier upward zero crossing and a later downward zero crossing in the composite respiration signal.

14. The method according to claim 6, further comprising computing a composite scaling factor for the composite respiration signal that normalizes the composite respiration signal.

15. The method according to claim 1, wherein defining the reference respiration signal using the plurality of non-driven impedance signals comprises defining either a strongest non-driven impedance signal of the plurality of impedance signals or a first principal component signal of the plurality of non-driven impedance signals as the reference respiration signal.

16. The method according to claim 15, wherein the strongest non-driven impedance signal of the plurality of non-driven impedance signals comprises a largest standard deviation signal of the plurality of non-driven impedance signals.

17. The method according to claim 15, wherein the strongest non-driven impedance signal of the plurality of non-driven impedance signals comprises identifying a highest amplitude signal of the plurality of non-driven impedance signals.

18. The method according to claim 15, wherein defining either the strongest non-driven impedance signal of the plurality of impedance signals or the first principal component signal of the plurality of non-driven impedance signals as the reference respiration signal comprises defining either the strongest impedance signal of the plurality of non-driven impedance signals or the first principal component signal of the plurality of non-driven impedance signals as the reference respiration signal according to correlation coefficients between the strongest impedance signal and the subset of the plurality of the non-driven impedance signals, on one hand, and between the first principal component signal and the subset of the plurality of the non-driven impedance signals, on the other hand.

19. An electroanatomical mapping system, comprising:
a respiration compensation module configured to:
receive a plurality of non-driven impedance signals from a plurality of patch electrodes;
define a reference respiration signal using the plurality of non-driven impedance signals;
for each non-driven impedance signal within a subset of the plurality of non-driven impedance signals:
compute a polarity value for the non-driven impedance signal; and
compute a scaling factor for the non-driven impedance signal; and
compute a composite respiration signal from the subset of the plurality of non-driven impedance signals by multiplying each non-driven impedance signal within the subset of the plurality of non-driven impedance signals by its corresponding polarity value and scaling factor, thereby computing a plurality of weighted non-driven impedance signals, and summing the plurality of weighted non-driven impedance signals.

20. The system according to claim 19, wherein the respiration compensation module defines the reference respiration signal as either a strongest non-driven impedance signal of the plurality of impedance signals or a first principal component signal of the plurality of non-driven impedance signals as the reference respiration signal.
